(19) United States
(12) Patent Application Publication
(10) Pub. No.: US 2019/0156938 A1
(43) Pub. Date: May 23, 2019

(54) SYSTEM, METHOD AND DATA MODEL FOR SECURE PRESCRIPTION MANAGEMENT

(71) Applicant: Michael Brunner, Keene, KY (US)

(72) Inventor: Michael Brunner, Keene, KY (US)

(21) Appl. No.: 16/196,102

(22) Filed: Nov. 20, 2018

Related U.S. Application Data
(60) Provisional application No. 62/588,702, filed on Nov. 20, 2017.

(51) Int. Cl. G16H 40/20; G16H 10/60; G16H 70/40; H04L 9/06

(52) U.S. Cl. CPC G16H 40/20; G16H 10/60; H04L 2209/38; H04L 9/0637; H04L 9/0643; G16H 70/40

(57) ABSTRACT

Methods, apparatus, media, and a data model for managing prescription records. A prescription request is received from a prescriber, the prescription request including prescription data describing a prescription and, optionally, a value in cryptographic tokens associated with the prescription data. The request is approved if the prescriber is an authorized prescriber. Prescription data is recorded as a prescription record. A cryptographic hash of at least some of the prescription data and auxiliary information is created as a unique identifier for the prescription and the hash, a prescriber ID, a pharmacy ID and a patient ID are recorded on a blockchain as an issue transaction corresponding to the prescription. An acceptance message is received indicating that a pharmacy is willing to fill the prescription. If the acceptance message is verified, an accept transaction is recorded on a blockchain corresponding to the prescription, the accept transaction including the hash.

### RELATED APPLICATION DATA

This application claims priority to U.S. Provisional Patent Application Ser. No. 62/588,702 filed on Nov. 20, 2017, entitled “Distributed Ledger Data Structure for Prescription Security and Fidelity”, the disclosure of which is incorporated herein in its entirety.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD

The present disclosure relates to a distributed ledger architecture, method and data structure for prescription secure prescription management.

### BACKGROUND

Prescription drug abuse, specifically of opioids, is possibly the most significant public health crisis of the 21st century. It has been estimated that 2 million adults over the age of 12 have a substance abuse disorder involving prescription pain relievers The American Society of Addiction Medicine has observed that drug overdose is the leading cause of accidental death in the United States, with almost half of the fatalities coming from prescription opioid abuse. Opioid Addiction 2016 Facts & Figures. (2017, Jan. 1): https://www.asam.org/docs/default-source/advocacy/opioid-addiction-disease-facts-figures.pdf. Leading projections indicate that up to half a million Americans could die from prescription opioid overdoses within the next 10 years. This death toll rivals the projected deaths from breast cancer and prostate cancer combined. Additionally, this is similar to the AIDS epidemic of the 1980's in death toll and scope. STAT forecast: Opioids could kill nearly 500,000 Americans in the next decade. (2017, Jun. 27) https://www.statnews.com/2017/06/27/opioid-deaths-forecast/. Drug overdose deaths in the United States in 2016 exceeded the deadliest car collision death toll year on record. Drug Deaths in America Are Rising Faster Than Ever (2017, Jun. 5) https://www.nytimes.com/interactive/2017/06/05/upshot/opioid-epidemic-drug-overdose-deaths-are-rising-faster-than-ever.html. Further, this crisis disproportionately affects the most vulnerable populations among us.

In an attempt to curb this growing public health crisis, the Center for Disease Control (CDC) has recently issued new opioid prescribing guidelines. “Guidelines for Prescribing Opioids for Chronic Pain” (2017, Aug. 29). Unfortunately, these guidelines are merely suggestions for proper prescribing practices. The rampant overprescribing of opioid pain medications, prescription fraud, and a system of documentation and prescription generation which fundamentally relies on trust between the patients, prescribers, and pharmacies are just a few of the contributing factors that have led to this epidemic.

Most often, opioid prescriptions are handwritten on paper and can be altered, outright forged, and/or duplicated. Pharmacies are forced to trust that the doctor is the one who physically wrote and signed the paper prescription often based on nothing more than professional judgment. Issues in interpreting handwritten prescriptions also cause medication errors and may result in prescriptions being filled improperly or incorrectly. Additionally, physicians are often unaware if the patient has received prescriptions from another doctor for the same medication, thereby doubling the patient's supply of medication. The current system for prescribing controlled substances is untrustworthy, in most instances untraceable, and unreliable.

The problem is multi-faceted. Careless or corrupt providers overprescribe opioids while abusers alter, forge, and duplicate paper prescriptions. Responsible doctors and pharmacists often have no way of knowing whether they're providing drugs to a legitimate user, or someone with illegal intentions. Traditional arrangements relying on trust and goodwill have become untraceable and unreliable. Laws, regulations, and professional guidelines have all failed to solve the problem.

### SUMMARY

One implementation is a computer implemented method for securely managing prescription records, the method comprising: receiving, from a prescriber computing platform, a prescription request, wherein the prescription request includes prescription data describing a prescription: approving the prescription request if the prescriber platform has been authenticated as corresponding to an authorized prescriber; recording the prescription data in a database as a prescription record, wherein the prescription record includes a prescriber ID, a pharmacy ID, a patient ID, and a medication; creating a cryptographic hash of at least some of the prescription data and auxiliary information to create a unique identifier for the prescription; recording the hash as an issue transaction corresponding to the prescription; receiving, from a pharmacy computing platform, an acceptance message indicating that the pharmacy platform is willing to fill the prescription; verifying the accept message if the pharmacy platform corresponds to the pharmacy ID and the pharmacy is an authorized pharmacy; and recording an accept transaction on a blockchain corresponding to the prescription, the accept transaction including the hash.

Another implementation is a computer system securely managing prescription records, the method comprising: at least one computer hardware processor; and at least one computer memory storing instructions thereon which, when executed by the at least one computer hardware processor, cause the at least one computer hardware processor to: receive, from a prescriber computing platform, a prescription request, wherein the prescription request includes prescription data describing a prescription; approve the prescription request if the prescriber platform has been authenticated as corresponding to an authorized prescriber; record the prescription data in a database as a prescription record, wherein the prescription record includes a prescriber ID, a pharmacy ID, a patient ID, and a medication; create a cryptographic hash of at least some of the prescription data and auxiliary information to create a unique identifier for the prescription; record the hash as an issue transaction corresponding to the prescription; receive, from a pharmacy computing platform, an acceptance message indicating that the pharmacy platform is willing to fill the prescription; verify the accept message if the pharmacy platform corresponds to the pharmacy ID and the pharmacy is an authorized pharmacy; and record an accept transaction on a blockchain corresponding to the prescription, the accept transaction including the hash.

### BRIEF DESCRIPTION OF THE DRAWING

Implementations are described in connection with the attached drawing in which:

FIG. 1 is a schematic diagram of a computer architecture in accordance with an implementation.

FIG. 2 is a messaging diagram in accordance with an implementation.

FIG. 3 is a flowchart of an example lifecycle of a prescription.

### DETAILED DESCRIPTION

Implementations disclosed herein provide a system for fundamentally changing the way medication, such as opioids, are prescribed and supplied by creating a secure and effective method and system for providers, pharmacists and patients. Implementations leverage the transparency and immutability of blockchain and Distributed Ledger Technology (DLT) (collectively referred to as “blockchain” herein).

A blockchain is an append only list of records, called blocks, which are linked using cryptography. Typically, each block contains a cryptographic hash of the previous block, a timestamp, and transaction data. A blockchain is typically managed by a peer-to-peer network collectively adhering to a protocol for inter-node communication and validating new blocks through a “consensus mechanism.” The blockchain is stored on multiple nodes of the network. Data cannot be altered retroactively without alteration of all subsequent blocks, which requires consensus of the network majority and extremely high computing power. Blockchain was disclosed by a party under the pseudonym of “Satoshi Nakamoto” in 2008 to serve as the public transaction ledger of the cryptocurrency bitcoin. User's transact on a blockchain using cryptographic “wallets”, a secure memory device having an address corresponding to the user and able to store cryptographic keys corresponding to transactions recorded on the blockchain.

Blockchain can use known public-key cryptography, also known as asymmetric cryptography, to verify identities of parties. Public key cryptography uses pairs of keys: public keys which may be disseminated widely, and private keys which are known only to the owner. This permits authentication, where the public key verifies that a holder of the paired private key sent the message, and encryption, where only the paired private key holder can decrypt the message encrypted with the public key. A party can encrypt a message using the receiver's public key. That encrypted message can only be decrypted with the receiver's private key. Executable logic, known as “smart contracts” can be immutably recorded on a blockchain and can be used to execute various functions in a predictable manner.

Implementations disclosed herein address the problems of prescription security and fidelity by leveraging blockchain technology by means of a novel system through use of which a novel data model is created, accessed, and monitored, as described in detail below. As an example of a suitable blockchain to be used by the system, the Ethereum blockchain can be used. The implementations provide a cryptographically secure, HIPAA compliant, end-to-end prescribing system. A prescription data structure is securely stored on a blockchain and can be associated with an amount of native tokens (referred to as “tokens” herein) that are specific to the system. The prescription data structure (sometimes referred to as “prescription” herein) includes prescription information (e.g., type of drug, patient, quantity, dose, pharmacy . . . ) and a unique transaction identifier to securely verify the prescription's origin from point of creation by a physician.

The physician accesses a distributed application (D-app), for example, in the physician's office, where the distributed application is used to generate a prescription. The physician signs the prescription, such signature being accomplished by a biometric signature means such as a fingerprint or facial recognition, such biometric signature means being paired to, e.g. derived from, the physician's private key on a network accessed by the distributed application. The prescription is signed, and a token amount from the physician's integrated wallet, which is holding a balance of native network tokens, is paired to the prescription data structure. The prescription is then transmitted to a pharmacy management system (referred to as “the system” herein). The status of the prescription is managed by the system and recorded on a blockchain to securely mange the lifecycle of each prescription.

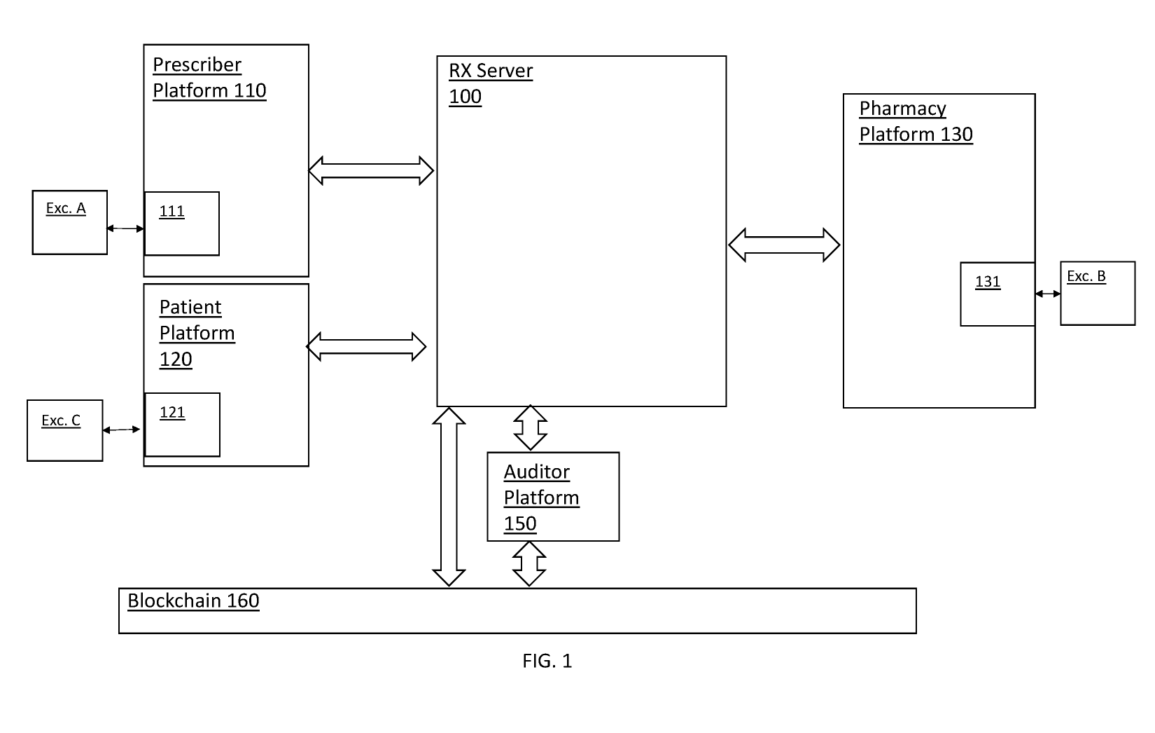

FIG. 1 illustrates the computer architecture of an implementation at a high level. Physicians, pharmacies, and patients can securely access the system. Physicians communicate with RX Server 100 of the prescription management system through prescriber computing platform 110 which includes a subscriber crypto wallet 111 and one or more client computing devices. As discussed above, the crypto wallet stores keys to transactions on a blockchain and thus effectively “stores” tokens belonging to the physician. Using prescriber computing platform 110, the physician will have the ability to view their own prescribing history and the prescription history of patients. Physicians will have the ability to issue new prescriptions by filling out the necessary auto-populated fields in an electronic form displayed on prescriber computing platform 110. The physician can specify the pharmacy that is to receive the prescription, or the prescription can be designated to be handled by any pharmacy that uses the system.

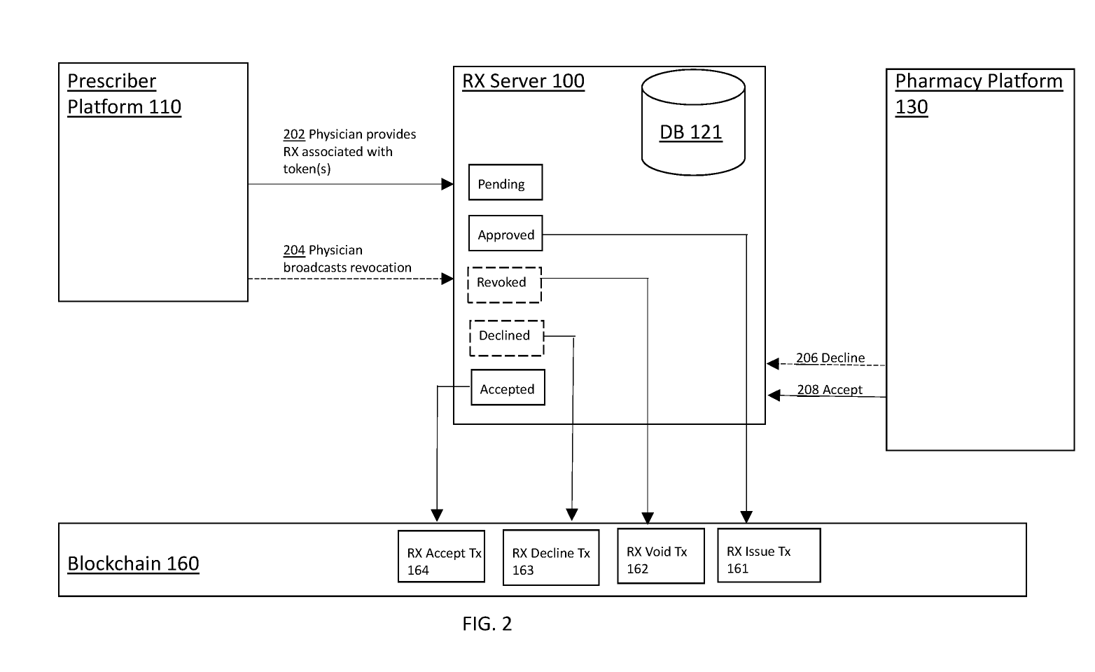

To issue a new prescription the physician may be required to pay a fee in tokens and the appropriate fractional or multiple number of tokens are associated with the specific prescription. The user interface of prescriber platform 110 allows the prescriber to view their available token balance and the required fee for creating the prescription. The prescription will be digitally signed by RX server 100. By signing the prescription, RX Server 100 will verify that the prescription is valid, and that the physician has been authenticated in a sufficient manner (depending on prescription type, applicable laws and any other appropriate considerations). When a prescription is issued by a physician, it is in a “pending” state, waiting for signature by RX Server 100. Once RX Server 100 digitally signs the prescription the signed prescription transaction is recorded on a blockchain 160 and is set into an “approved” state, as described in more detail below with reference to FIG. 2.

Physicians can view a list of their own prescription history which will include the current prescription's status: pending, approved, revoked, declined, or accepted. Physicians will also be able to see if the patient attempted to use his/her prescription at more than one pharmacy. If a prescription is in a pending or approved state, the physician may revoke it which would cause it to enter a revoked state. The revoke transaction is also recorded on the blockchain, as described below. If a prescription is revoked, or if it is declined by the pharmacy, the tokens will be returned to the physician as tokens to be issued. The physician will be able to reissue revoked prescriptions and select a pharmacy to send the prescription to, which may be the previous pharmacy or a different pharmacy.

RX Server 100 provides a set of prescriber wallet addresses to be used only by Physicians. Each physician will own one or more of these addresses. RX Server 100 will assign these addresses using a smart contract function which then records these addresses on a blockchain. These addresses are the only addresses that can be used to issue prescriptions. These addresses cannot directly transfer tokens to another arbitrary address. The tokens can only be transferred from these addresses in connection with issuing prescriptions.

Pharmacies communicate with RX server 100 through pharmacy platform 130 including a pharmacy crypto wallet 131 and one or more computing devices executing a D-app. RX server 100 can presents pharmacies with a queue of approved prescriptions. For each prescription, the pharmacy will be given the prescription information, and the ability to view a list of the patient's prescription history. They will have the option to accept or decline each prescription.

More specifically, for each prescription, the designated pharmacy will gain access to the prescription information (RX data structure) stored on RX Server 100 and, if the pharmacy accepts the prescription, will gain ownership of any token(s) paired with the prescription. If the pharmacy rejects the prescription, the tokens are transferred back to the crypto wallet of the physician. If the pharmacy accepts the prescription, the token is transferred to the pharmacy's crypto wallet 131. The use of tokens is optional however and transactions can be made and recorded without the transfer of tokens. In an example, a pharmacy employee opens the D-App running on pharmacy platform 130 to gain access to a prescription from the network and signs a confirmation of receipt of the prescription. The confirmation signature can be accomplished by a biometric signature mechanism such as a fingerprint or facial recognition. The prescription can then be printed on security paper with a corresponding QR code, having a transaction ID of the prescription recorded therein, which provides a link to the system. Use of the QR code is discussed in greater detail below.

Patients communicate with RX Server 100 through patient platform 120 which includes crypto wallet 121 and one or more computing devices executing a D-app. For example, patient platform 120 can be running on a mobile phone belonging to the patient. Patients can view their prescription history after login. Patients can hold tokens in cryptocurrency wallet 121. Since tokens have a market exchange rate, they can be used by patients to pay their co-pay payments at the pharmacy. Patients pay with tokens by scanning a QR code or communicating the transaction ID (and thus the corresponding token amount) to wallet 131 of pharmacy platform 130 through NFC functionality or in any other manner. A patient or patient designee can also scan a QR code corresponding to a prescription to verify that the prescription has been filled by a particular pharmacy and thereby control which pharmacy takes ownership of any tokens that may be associated with the prescription as payment for the prescription.

Third party auditors, or any other entity requiring access, such as a government regulator which is legally required to audit any such transactions, can be granted access to the blockchain transactions through auditor platform 150 which can be authorized to read relevant information directly from blockchain 160 and/or through RX server 100. Examples of auditors could include the U.S. DEA or the healthcare organizations to which the physicians belong.

Prescriptions cannot be duplicated in any manner. Prescriptions are issued using a smart contract function which is described in greater detail below. RX server 100 will record a unique identifier for each prescription on blockchain 160. Each uniquely identifiable prescription will only exist once in the blockchain 160: the prescription issuance will be rejected if a prescription having the identifier already exists on the blockchain. In other words, a prescription can only be accepted once and can only be accepted if the prescription is approved and has not been revoked or declined. When a prescription is requested on the platform, it is given a pending status. RX server 100 will call a contract function to move the status to approved and write the prescription to the blockchain. Pharmacies can only accept approved and non-revoked prescriptions as described in detail below.

Optionally a pharmacy can sell the tokens on the open market back to physicians' offices or others who require use of such tokens. As noted above, a physician optionally is required to possess tokens in order to pay for transmission of the physician's prescriptions using the D-app. In such a case, transmission of a prescription by a physician and acceptance thereof by a pharmacy constitutes a micro-transaction of a token (or a fraction of a token or multiple tokens) from a physician to a pharmacy. Upon accepting a prescription, a pharmacy will redeem the tokens paid by the physician and thereby make them freely spendable. When a prescription is accepted, the number of tokens associated with the prescription will be added to the pharmacy's wallet and be debited from the physician's wallet.

Prescriptions can only be issued by authorized physicians and accepted by authorized pharmacies. The system will verify the eligibility of physicians and pharmacies before they can use the system and before they are allowed to create accounts on the system. The system administrator will determine what patients a physician may issue prescriptions for and what prescriptions a pharmacy may accept or decline. RX server 100 will call an Approve function in the smart contract to make a prescription valid and available for acceptance by a pharmacy. The system will only do this if the physician has the authority to issue such a prescription. The address of a pharmacy that should accept or decline a prescription can be included in the issuance transaction, and only the address corresponding to this pharmacy will be able to view and accept, or decline, the transaction. All prescriptions on the system will be identifiable on blockchain 160. Further, blockchain 160 will record an eventually consistent (after sufficient confirmations) state of prescriptions that can be universally agreed upon.

Relevant prescription information will be made available to physicians and pharmacies. Patients will be able to view their own prescription history. Prescription information will be hidden from the general public. Prescription data can be stored on secure servers and will be provided to authenticated users that are authorized to view such data. In some implementations, only cryptographic hashes of this data will be stored on blockchain 160, alongside some limited information that pseudo-anonymously identifies physicians, patients and pharmacies, the token amounts, and the prescription status. This association can occur on the application level, or on the blockchain level. When at the application level, auditors will request the data from the application, not the blockchain.

Cryptographic hashes allow someone with the prescription data to recreate the hash and verify the validity, state, and version of the prescription. However, it does not allow anyone with the hash to compute the prescription data. These hashes can be associated with the address of the physician that issued it, the pharmacy that is allowed to accept it, and a confidential patient ID. Therefore, it is possible for those who can associate these addresses or IDs with particular doctors, pharmacies, and patients to know who was involved with a particular prescription hash. As noted above this association can be made at RX Server 100, i.e., “off-chain” and thus can be restricted to only be divulged to auditor authorities, or other entitled parties, under appropriate conditions. If the personal identity of an address or ID is unknown, it is not possible to know which physician or pharmacy issued or filled a prescription and for which patient.

If a Physician, patient or pharmacy interacts with another third-party application, such as an exchange (Exc. A, Exc. B and/or Exc. C in FIG. 1) using their tokens, then that third-party application can possibly reconstruct partially the identities of the interacting participants. However, these third-party applications won't be able to reconstruct the prescription data. The prescription data itself can be stored securely on RX server 100. Authentication of physicians, pharmacies, patients, and auditors will be required before they are given access to this data. All communication between users and the RX server 100 can be secured using strong TLS security. Patient IDs can be derived from a unique string, such as the patient's Social Security Number, and can include a secret salt. This salt will not be public and will only be shown to the auditors. These IDs can be solely for internal application level unique identifiers and not used for creating any type of asymmetric key pair.

Auditors can be permitted to view all prescription data on RX server 100 corresponding to the prescription hashes on the blockchain. Auditors will be able to verify the identity of pharmacies. All pharmacies will digitally sign their prescriptions (as blockchain transactions) with the key associated with their wallet address. Auditors will be provided these names and signatures in order to show the identity of a pharmacy with ownership of a particular wallet address.

As noted above a QR code can be made that contains data that can be used to verify a particular prescription has been accepted by a pharmacy and identify which pharmacy. More specifically, the QR code can contain the prescription hash and an access code to download the prescription data from RX server 100. Blockchain 160 can contain the prescription information for the given hash that should correspond to the downloaded information. This would demonstrate that the prescription has been accepted. The access code will be used to additionally download a message that contains the name of the pharmacy. This message will be digitally signed by the key that was used to sign the prescription acceptance transaction by the pharmacy. This signature will be downloaded alongside the message and will be used to verify that the name coincides with the pharmacy that agreed to the prescription. A pharmacy must digitally sign its name with a wallet address before the platform will approve a prescription specifying that address on the blockchain. By approving a prescription and by providing the name in response to the QR access code, the platform expresses that the pharmacy name associated with the pharmacy wallet address has been approved. All communications can be over HTTPS with a verified TLS certificate.

The token can use, for example, an Ethereum token standard. ERC 20 is the most common Ethereum token standard. Any application or other smart contract can interact with a standard token in a standard manner without a need of knowing other details about the token. For instance, crypto wallet developers can avoid custom development and integrations to add new tokens. All they need to know is the Ethereum Token address that implements the standard. Other Ethereum token standards include ERC 223, ERC721, ERC-621, and ERC-827.

Workflow in the system can be controlled in a predefined and trusted manner using smart contracts executing on blockchain 160 or other logic executed by RX server 100. The smart contracts can include a “logic contract” that implements the logic of the application. It will contain a system owner address that will be the entry contract address. Only this address can be used to call the contract implementations. This contract will be replaceable only by the system governing body. The logic contract can provide the implementation of the current contract functions and can reference storage contracts. The logic contract serves as the controller to the storage contracts. Storage can be reused and upgraded through the use of separated storage contracts noted above. An “entry contract” will store a reference to the current logic contract and be the first point of contact for clients accessing the application. These contracts are described in greater detail below.

FIG. 2 illustrates the life-cycle of a single prescription in accordance with an implementation. A physician that has access to the RX platform, through prescriber platform 110, can issue a new prescription for any patient for whom they are authorized. This is accomplished by providing all the necessary prescription details through a user interface of the prescriber platform using prescriber wallet addresses that are set by the RX platform as noted above. The prescription details can be cryptographically hashed into a 256-bit number. Cryptographically secure hash functions are one-way functions. They are built so that it is practically impossible to reproduce data or produce some other data that corresponds to a particular hash without first knowing that data. Theoretically, hash collisions exist where two pieces of data conform to the same hash, but it is infeasible to find these. Therefore, it is pragmatically impossible to reverse this hash back into the prescription details, and it is pragmatically impossible to create the same hash with another prescription. The same hash cannot be used more than once, i.e. two or more identical prescriptions are not allowed. This can be accomplished by including substantially unique strings, such as serial numbers or time stamps, in each prescription record. Therefore, even a prescription from the same doctor to the same patient for the same medication will have a different hash due to the appended strings. The prescription information and association with tokens is transmitted to the RX server at 202. The physician must authenticate the prescription data using their own account within the platform. For example, authentication can be accomplished with a 2-factor authentication mechanism.

More specifically, an Issue function issues a new prescription based on the physician requests. An Rx data structure will be filled with the values for pharmacy ID, patient ID, and other information, such as, patient, drug type, dosage, and token amount, noted above. The physician corresponding to the Physician ID in the Rx data structure will be the sender. The issue time will be set to the current block timestamp on blockchain 160. The status will be set to Pending by RX server 100. In one example, when a prescription is issued by a physician, the physician must provide the following information:
Full patient name
Patient date of birth
Patient home address
Date that the prescription is written
Drug name and strength
Instructions for taking drug
Quantity supplied
Number of refills
Physician name
Physician DEA number
Physician address

The function will fail if the address does not map to a valid prescriber address. Upon failure, no state changes will be made. The initial status of the prescription, after transmission to RX Server 100, is Pending. Upon a physician creating an allowable prescription, the platform will change the status to Approved if specified conditions are satisfied. More specifically, an Approve function moves the status of the prescription to the status Approved if:
the physician that issues the prescription is authorized to do so;
the physician has signed the prescription data using a biometric authentication device;
the prescription hash corresponds to a valid prescription on the RX server;
the patient ID is valid; and
the specified pharmacy address corresponds to an approved pharmacy that is allowed to accept prescriptions for the particular patient.

Upon successful approval, the prescription hash will be stored on blockchain 160, as RX issue transaction 161, and the prescription hash will be mapped to the off-chain prescription data stored in database 121 of RX server 100. The hash will be stored in a list of mapped RX data in database 121 and will be added to the end of the following lists in DB 121: 1) a list of approved prescriptions for the receiving pharmacy; 2) a list of approved prescriptions for the patient designated in the prescription; and 3) A list of patient history for that patient and that physician. The deadline in the RX data structure will be set to the deadline argument in the prescription record. RX issue transaction is a data record that includes the prescription hash and identifiers for the corresponding physician, pharmacy, and patient.

As noted above, at this point, the physician may optionally broadcast a revoke request, at 204, that prevents the pharmacy from accepting the prescription, and voids the prescription. In the case of such an event, the prescription will enter a Revoked status and the tokens associated with the prescription will be redeemable by the physician's prescriber address. The revoked status can be recorded on blockchain 160, as RX void transaction 162, to effectively void the prescription. Revocation will only be successful if the revoke message was sent by the issuing physician address, the prescription exists, and if the prescription is in the Pending or Approved state. Upon success, if the prescription was in the Approved state, the RX hash will be removed from the following lists stored in database 121: 1) the list of prescriptions for the designated pharmacy; 2) the approved field inside the prescription list for the patient; and 3) the RX list for the pharmacy. Upon success of a revoke function, the prescription will be moved to the end of the history field inside prescription list for the patient.

Assuming that the prescription has not been revoked by the physician, the pharmacy that owns the address in the prescription data may broadcast a decline message, at 206, for an Approved prescription to express that they are unwilling and/or unable to dispense the medication. In such as case and RX declined transaction 163 will be recorded on blockchain 160. If the prescription is in the Declined status, the tokens associated with the prescription will be redeemable by the physician's prescriber address/wallet. At this point, the prescription becomes void and the physician may have to reissue another prescription to another pharmacy, in the same manner described above, if the patient requires the medication.

More specifically, a Decline function executing on RX serve 100 changes the status of a prescription given by the corresponding hash to a Declined status when the pharmacy address found within in the message corresponds to the pharmacy designated in the Rx data structure, the prescription exists, and if the prescription is in the Approved state. Further the prescription hash will be removed from the following lists stored in database 121: 1) Prescription list for the receiving pharmacy; 2) the approved field inside the prescription list for the patient; and 3) the prescription list for the pharmacy and patient. Also, the prescription will be moved to the end of the history field inside prescription list for the patient.

Alternatively, the pharmacy that owns the address in the prescription may transmit an accept message, at 208, to accept an Approved prescription. The pharmacy should wait for a confirmation or a number of confirmations on the blockchain to ensure that the transaction is sufficiently entrenched and that there is little risk of there being a conflicting revocation transaction by the physician. This wait can be enforced as a mandatory time period between approval and acceptance, for example. Accept message 208 will result in the prescription being set to the Accepted state. Once the prescription is in the Accepted state, RX accept transaction 164 is written to blockchain 160 and the medication may be provided to the patient by the pharmacy. Further, a filled transaction can be recorded on blockchain 160 once a prescription had been filled.

More specifically, an Accept function changes the status of the prescription to Accepted. This will only be successful if the accept message was sent by the pharmacy address found in the prescription, the prescription hash exists on blockchain 160, the prescription is in the Approved state, and the current block timestamp (time at acceptance is before the prescription's deadline/expiration date. Otherwise the transaction will fail, and no state changes will be made. An acceptance transaction will include the prescription hash and the Ethereum address of the pharmacy that should accept or decline the prescription. The transaction will also include a pseudo-anonymous patient identifier to verify that the prescription was made for a particular patient.

Upon success, the prescription hash will be removed from the following lists stored in database 121: 1) The prescription list for the receiving pharmacy; 2) the approved field inside prescription list for the patient; and 3) the prescription list for the pharmacy and patient. Upon changing to the Accepted state, the prescription hash will be added to the following lists stored in database 121: 1) the prescription list for the receiving pharmacy and the patient; and 2) the accepted field inside prescription list for the patient. Also, the prescription will be moved to the end of the history field inside prescription list for the patient and a Time Accepted field in the Rx data structure will be set to the current block timestamp (time at change to Accepted).

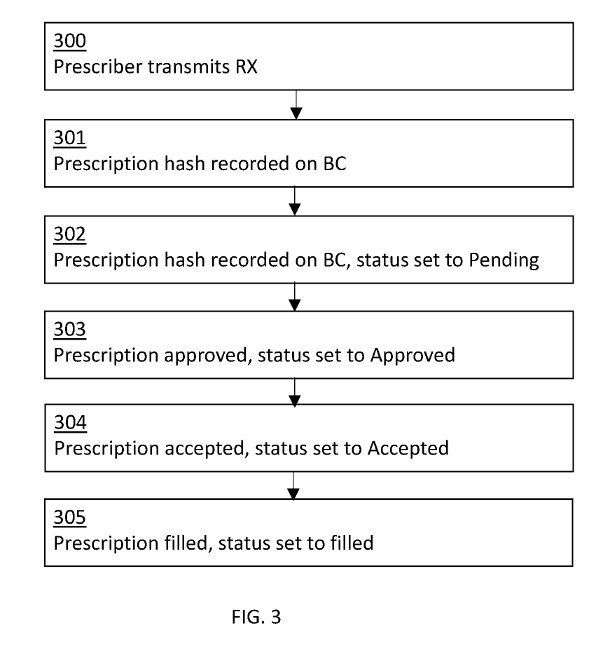

For clarity, a simple example of the workflow described above, assuming that the prescription is approved, not revoked by the physician, and accepted by the pharmacy, is summarized below with reference to FIG. 3. At step 300, the prescriber physician transmits a prescription to the system. At 301, the prescription record is hashed, and the hash is recorded on a blockchain. At 302 status of the prescription is set to Pending. at 303, the prescription is approved by the system in the manner described above and status is set to approved. At 304, the prescription is accepted by the designated pharmacy and the status is set to accepted. at 305, the prescription is filled, and status of the prescription is set to filled. At each step, the appropriate system records are updated, and transactions can be recorded to a blockchain as described above.

Other examples of functions that can execute on RX server 100 or as smart contracts on the blockchain are:
setPrice: This function sets the required token price for a prescription to be issued. The sender address must correspond to the platform.
getPhysicianHistory: This function gets a prescription for a physician address. StatusPending prescriptions are not included. The first prescription is obtained using 0 as the index. Physician Prescription List.
getPharmacyQueue: This function gets a Approved status prescriptions for a pharmacy address. The prescriptions are obtained in reverse order from the Pharmacy Prescription List.
getAcceptedForPatient: This function gets all Accepted status prescriptions for a patient ID. The prescriptions are obtained in reverse order from an accepted array from the Patient Prescription List.
getApprovedForPatient: This function gets all Approved status prescriptions for a patient ID. The prescriptions are obtained in reverse order from the approved Patient Prescription List.
getPatientHistory: This function gets a prescription for a patient ID. Pending prescriptions are not included. The prescriptions are obtained in reverse order from the history of the Patient Prescription List.
getPhysicianPatientHistory: This function gets a prescription for a physician and patient ID pair. Pending prescriptions are not included. The prescriptions are obtained in reverse order from the Physician-Patient History List.
getAcceptedForPharmacyPatient: This function gets all Accepted prescriptions for a pharmacy address and patient ID pair. The prescriptions are obtained in reverse order from an array in the Pharmacy-Patient List.
getPharmacyPatientQueue: This function gets all Approved prescriptions for a pharmacy address and patient ID pair. The prescriptions are obtained in reverse order from the Pharmacy-Patient List.
getRx: This function gets the Rx structure corresponding to a prescription hash.
getNumRxs: This function obtains the total number of prescriptions. Useful for auditors to iterate through all of the prescriptions.
setPrescriberAddress: This function adds the address to the prescriber address mapping database. The requestor address must correspond to the owner of the platform.

All of the status logic above can be accomplished logic internal to RX server 100 and/or by smart contracts recorded on blockchain 160. In the example above, each status changes was recorded in blockchain 160. However, status can be tracked by RX server 100 without recording individual status changes as transactions on blockchain 160.

As noted above, auditors might require access to some or all of the information available on the platform. On blockchain 160, auditors can be given access to:
The prescription status
The address of the physician that issued a prescription
The address of the pharmacy specified to fill the prescription
The ID for the prescription
The application that signed the prescription (or specific transaction related thereto) and committed it to the blockchain

This information will be tied to a prescription hash that corresponds to a particular prescription. Auditors will be provided a full list of prescription data by the system that corresponds exactly to the list of prescription hashes up-to a particular point in the blockchain state. This data will include all the required information for a prescription. Auditors may also need to know the identities of the patients for each patient ID. The patient identities must not be vulnerable to deception. A patient ID must verifiably correspond to one patient. As an example, the patient IDs can be salted hashes of the patient's Social Security Number (SSN). The Auditors will be given the SSNs of all patients on the system alongside the hash salt so that they can verify that there is only one patient ID for each patient, and that physicians, and pharmacies can receive the full prescription history for a patient from a single ID. Auditors will then also be able to audit the prescription history for each patient as is necessary for them to do so.

When an Ethereum address is generated for a pharmacy, the key for that address is used to sign the name and address of the pharmacy. The auditor can be provided the details of each pharmacy alongside these digital signatures, so that the auditor knows the claimed identity of pharmacies that accept prescriptions. The physician's details will be included in the prescription data itself.

Auditors can also be provided with the digital signatures of the prescription data made by authentication devices. Physicians will use these devices to verify their personal identity when issuing prescriptions. Auditors will need to know which public keys are associated with which physician. If they need to verify this with any particular physician, they can oversee the physician using the device to provide proof that the device can sign for a particular public key.

As noted above, physicians will be authenitcated when issuing prescriptions. Examples authentication mechanisms could include:
Secure hardware devices
Software based 2 factor authentications
Software or hardware based multi-factor authentications
Software based 2^{nd} device login prompt
QR code based session login through secondary device

This additional authentication can be used for two purposes: 1) the platform will require authentication before sending an “approve” transaction for a particular prescription and 2) Auditors will verify the authentication when required, to verify that prescriptions were indeed issued by a particular physician. The additional authentication response should be valid only once and tied exclusively to one prescription. Otherwise the response could be used to arbitrarily authenticate under the particular identity through replay attacks. Only the owner of an authentication device (the physician) should be able to provide a successful authentication response. Not even the system should be able to construct successful responses. The response should not disclose any sensitive information. Digital signatures can fulfil these requirements. If only the physician can utilize a private key, then only the physician can provide a successful response via a digital signature. Secure digital signatures do not disclose the private key, and do not allow anyone with access to a signature to produce any other valid signatures.

As noted above, prescription data can be displayed in multiple lists. The term “list”, as used herein, refers to any type of storage mechanism or structure, such as a table, a text document, a relational database or the like. The lists can be stored in one structure or in many different structures. Therefore, the term “database”, as used herein, refers to any collection of data in any form on a single device or multiple devices. Different types of users will be able to view each list according to their role and need. For example, auditors might be able to view all lists. Each list will contain prescriptions that conform to specified criteria, e.g. belonging to a particular patient or being in a particular status. Prescriptions can be added to the end of lists or moved (“bumped”) to the top of the list under certain conditions. The contents of each list can be updated and verified by the smart contracts. For example, a Physician History list can be viewed only be a specific physician and include prescriptions issued by that physician. Prescription records can be moved the end of the list when the prescription is issued. Of course, various other lists can be created for various viewers and can include records satisfying various parameters as needed.

As noted above, various messages containing data structures are exchanged and recorded. Enumerated types (referred to as “enum” herein), i.e. data types including a set of named values called elements are used by the disclosed implementations. As noted above, status is used to record the current state of a prescription and a Status data structure includes the elements {StatusPending, StatusApproved, StatusRevoked, StatusDeclined, StatusAccepted}. Each status is described below.
StatusPending: Issued by a physician, but not approved by BlockMedx yet.
StatusApproved: Approved by the platform and available for acceptance by a pharmacy.
StatusRevoked: Revoked by the physician and unavailable for acceptance by the pharmacy.
StatusDeclined: Declined by the pharmacy.
StatusAccepted: Accepted by the pharmacy.

The Rx data structure contains the prescription data and can include the elements {address pharmacy; address physician; Status status; bytes32 patientID; uint256 mdxValue; uint timeIssued; uint timeAccepted; uint deadline;};

Each element is described below:
pharmacy: The address of the pharmacy that is allowed to accept the prescription and redeem the tokens.
physician: The address of the issuing physician.
status: The current status of the prescription.
patientID: The unique ID of the patient that the prescription is made for.
mdxValue: This is an optional element indicating the amount of token units that are optionally locked up by this prescription and redeemable by the chosen pharmacy.
timeIssued: The timestamp of the block where the issuing transaction was included.
timeAccepted: The timestamp of the block of the accept transaction, or 0 if not yet accepted.
deadline: The timestamp from which a prescription can no longer be accepted.

The data structure ListNode is used to iterate through a prescription list and includes the elements {uint32 prev; uint32 next;}. The data strucutre RxList contains a bidirectional iterable list of prescription hashes. Prescription hashes can be added to the end, and prescription hashes can be removed from the list.

The data structure RxList (Prescription List) contains the elements {mapping(uint32=>ListNode) nodes; mapping(uint32=>bytes32) rxs;}. This data structure can be implemented as a circular linked list, using a sentinel ListNode, in similar fashion to an existing implementation known as LibCLL4. Element indexes start at 1. The ListNode at index 0, is a special sentinel node that refers to no actual elements in the list, but stores the first element of the list in next, and the last element of the list in prev. If there are no elements in the list, then next and prev in the sentinel will store 0, and thus point to itself. Iteration starts from the sentinel node. From this node, each element in the list can be iterated by following the next index for forward iteration and the prev index for reverse iteration. The end of the list is indicated by returning to the sentinel node at index 0. Prescription hashes are obtained from rxs using the same index as the node. During iteration, a node may be accessed which was deleted. In this case, it will be possible to check if the node was deleted by the rxs value, which would be set to 0. Any such element will be skipped in an iteration. Elements are added to the list by creating a new ListNode. The index of this node is the index of the previous node, plus one. The next index is set to the sentinel index (0). The prev index in the sentinel node is set to the index of the added node. The prev index is set to the index of the node that was previously at the end. The next index of the previous node is set to the new node. The prescription hash will be added to rxs at the same index as the node. Elements are removed from the list by setting the next node's prev to the prev of the removed node, and the previous node's next to the next of the removed node. The rxs of the element is mapped to 0, indicating that it is deleted.

The data structure PatientRxLists (Patient Prescription List) includes the elements {bytes32[ ] accepted; RxList approved; RxList history;}. This structure will contain lists of prescriptions for a particular patient. the element accepted will be for Accepted prescriptions. The prescriptions will be ordered by the sequence in which they were accepted. Once a prescription has been accepted, it cannot exit that state. Therefore, the array is a push-only structure. The element approved will be for Approved prescriptions. The element history will contain all patient prescriptions that are not Pending. Other lists noted above can be constructed in a similar manner.

Of course, various state variables are used by the platform. All state variables can be distinguished from other variables by being prefixed with “s”. Examples of state variables used in implementations are provided below:
sRxs: mapping(bytes32=>Rx) sRxs; This will map the 256-bit prescription hashes to the on-chain prescription data.
sPhysiciansRxLists: mapping(address=>RxList) sPhysiciansRxLists; This will map a physician's address to a list of prescriptions that they have issued and are any status.
sPharmacysQueueRxLists: mapping(address=>RxList) sPharmacysQueueRxLists; This will map a pharmacy's address to a list of prescriptions that are currently in the StatusApproved state and are destined for the pharmacy.
sPatientsRxLists: mapping(bytes32=>PatientRxLists) sPatientsRxLists; This will map patient IDs to an object containing lists for the patient's prescriptions.
sPhysiciansPatientsHistoryRxLists: mapping(address=>mapping(bytes32=>RxList)) sPhysiciansPatientsHistoryRxLists; This will map physician addresses to mappings that map patient IDs to a list of prescription history for the patient and physician. The prescriptions are not StatusPending.
sPharmacysPatientsAcceptedRxLists: mapping(address=>mapping(bytes32=>bytes32[ ])) sPharmacysPatientsAcceptedRxLists; This will map pharmacy addresses to mappings that map patient IDs to an array of StatusAccepted prescriptions for the patient and pharmacy.
sPharmacysPatientsQueueRxLists: mapping(address=>mapping(bytes32=>RxList)) sPharmacysPatientsQueueRxLists; This will map pharmacy addresses to mappings that map patient Ids to a list of StatusApproved prescriptions for the patient and pharmacy.
sMdxRxPrice: uint256 sMdxRxPrice; In the case of tokens associated with the prescription, this this optional state variable will be the current required MDX units required for a prescription.
sRxList: bytes32[ ] public sRxList; This will be a public array of all the prescriptions, regardless of status, so that auditors can access them all. This will be a push only array. An auditor will be able to begin reading prescriptions from the same index that they last finished with.
sPrescriberAddresses: mapping(=>bool) sPrescriberAddresses; This will map addresses to whether or not they are considered a prescriber address. If an address maps to true then it is considered a prescriber address and should be handled as such, ie. allowing issuance of prescriptions.

Off-chain data can be stored securely on RX server 100 or in other devices, with multiple layers of redundancy. Data structures that are hashed, can be encoded first using a canonical platform-independent encoding format, such as DER (Distinguished Encoding Rules) or CANONICAL-PER (Canonical Packed Encoding Rules).

As noted above, at step 202 of FIG. 2, the physician provides prescription information. Prescription information can be encoded and then hashed, using SHA-256 for example, into the prescription hash that will be recorded on the blockchain. RX server 100 will also store an access code to be provided on prescriptions within the QR code, used for validation of the prescription. Full and unique pharmacy names associated to each pharmacy wallet address will be stored by RX server 100 in association with the corresponding address. ECDSA signatures of these details will be made using the private keys associated with the pharmacy addresses. These signatures will also be stored by RX server 100 in association with the pharmacy details to be provided in response to prescription QR access codes, and to auditors. Usernames and hashed passwords can be stored on the RX server 100 used for physician, pharmacy, and patient access. Passwords can be hashed using bcrypt. RX server 100 can also store other relevant user data such as session IDs.

As noted above, the various functions of the platform can be accomplished by smart contracts executing on the blockchain and/or by more conventional business logic executing on RX server 100. Examples of the logic of various specific functions are discussed below. The Examples are written in the Solidity language for the Ethereum blockchain using the ERC20 Token standard. The example contracts are stored as .sol (solidity) files and are discussed individually below.

Application.sol:

This contract serves as the entry point to the platform application and provides an ERC20 compliant token. Application.sol uses an underlying ‘Logic’ contract that can be replaced. The application.sol contract inherits from ‘Owned’ and ‘ERC20’. It provides the ERC20 token interface and contains an underlying ‘Logic’ contract which implements these functions. The ‘Application’ contract can also upgrade the underlying ‘Logic’ contract and extend storage using the ‘installLogic( )’ function. When a new logic function is installed, the previous ‘Logic’ contract is deactivated (“killed”) using the ‘kill( )’ function. The new ‘Logic’ contract will be made the writer of all of the ‘Store’ contracts, including the new one if provided. The new ‘Logic’ contract is initialised using the ‘init( )’ function. After this function is called, ‘getLatestLogic( )’ will return the new ‘Logic’ contract, and the ERC20 function implementations shall be provided by the new ‘Logic’ contract. The solidity code for the application.sol contract is set forth below.

```
// Copyright 2017 BlockMedx LLC.
pragma solidity \hat{ }0.4.18;
import “./owned.sol”;
import “./logic.sol”;
import “./store.sol”;
contract Application is owned {
  logic private sLogic;
  store[ ] private sStores;
  constructor(address owner) owned(owner) public { }
  function installLogic(logic newLogic, store newStore) external
onlyOwner {
    // Destroy previous logic contract
    if (sLogic != address(0))
      sLogic.kill(msg.sender);
    // Require that the logic contract is owned by Application
    require(newLogic.isOwner(this));
    if (newStore != address(0)) {
      // Push the new store to the array, ensuring it is owned by
Application
      require(newStore.isOwner(this));
      sStores.push(newStore);
    }
    // Go through all storage contracts and update the writer to the
    // new logic contract
    for (uint x = 0; x < sStores.length; x++)
      sStores[x].setWriter(newLogic);
    // Initialise the new logic contract
    newLogic.init( );
    // Set new logic contract
    sLogic = newLogic;
  }
  function getLatestLogic( ) external view returns (logic) {
    return sLogic;
  }
  function getOwner( ) external view returns (address) {
    return sOwner;
  }
}
```

Logic.sol: This is an abstract contract that contains required functions for providing the platform application logic. The solidity code is set for the below.

```
// Copyright 2017 BlockMedx LLC.
pragma solidity \hat{ }0.4.18;
import “./Application.sol”;
import “./owned.sol”;
import “./mortal.sol”;
contract logic is mortal {
  constructor(address owner) mortal(owner) public { }
  function init( ) external;
  function isAppOwner(address addr) public view returns (bool) {
    return addr == Application(sOwner).getOwner( );
  }
  modifier onlyAppOwner( ) {
    require(isAppOwner(msg.sender));
    _;
  }
  function version( ) public view returns (uint16 major, uint16 minor,
uint16 patch);
}
```

ERC20.sol:

This is an abstract contract for ERC20 functions and events. The solidity code is set for the below.

```
// Copyright 2017 BlockMedx LLC.
pragma solidity \hat{ }0.4.18;
contract ERC20 {
  function name( ) public view returns (string);
  function symbol( ) public view returns (string);
  function decimals( ) public view returns (uint8);
  function totalSupply( ) public view returns (uint256);
  function balanceOf(address who) public view returns (uint256);
  function allowance(address owner, address spender) public view
returns (uint256);
  function transfer(address to, uint256 value) public returns (bool);
  function transferFrom(address from, address to, uint256 value) public
returns (bool);
  function approve(address spender, uint256 value) public returns (bool);
  event Transfer(address indexed _from, address indexed _to, uint256
_value);
  event Approval(address indexed _owner, address indexed _spender,
uint256 _value);
}
```

Owned.sol: This contract can restrict access to certain functions and data to the owner of the contract. The solidity code is set forth below.

```
// Copyright 2017 BlockMedx LLC.
pragma solidity \hat{ }0.4.18;
contract owned {
  address internal sOwner;
  constructor(address owner) public {
    sOwner = owner;
  }
  function isOwner(address addr) public view returns (bool) {
    return addr == sOwner;
  }
  modifier onlyOwner( ) {
    require(isOwner(msg.sender));
    _;
  }
  function transferOwnership(address newOwner) external onlyOwner {
    sOwner = newOwner;
  }
}
```

Store.sol:

This contract has a writer and can restrict access to certain functions to that writer. The solidity codes is set forth below.

```
// Copyright 2017 BlockMedx LLC.
pragma solidity \hat{ }0.4.18;
import “./owned.sol”;
contract store is owned {
  address private sWriter;
  constructor(address owner) owned(owner) public { }
  function isWriter(address addr) public view returns (bool) {
    return addr == sWriter;
  }
  modifier onlyWriter( ) {
    require(isWriter(msg.sender));
    _;
  }
  function setWriter(address newWriter) external onlyOwner {
    sWriter = newWriter;
  }
}
```

Mortal.sol:

This contract extends owned.sol to allow the contract to execute ‘selfdestruct( )’ by the owner. The solidity code is set forth below.

```
// Copyright 2017 BlockMedx LLC.
pragma solidity \hat{ }0.4.18;
import “./owned.sol”;
contract mortal is owned {
  constructor(address owner) owned(owner) public { }
  function kill(address recipient) public onlyOwner {
    selfdestruct(recipient);
  }
}
```

A number of storage contracts can be used to hold data, i.e. the lists noted above, for the application. Each storage contract can contain an owner address, and a controller addresses. The owner address can change the controller address, or transfer ownership to another address. The controller address has write access to the storage. There can be one storage contract to store the token data, and another to store the initial state variables. Having multiple storage contracts, allows for extensible storage.

The logic contract can be changed/upgraded by calling a function named upgradeLogicContract in the entry. This function can only be called by the system owner address. The function will update the address of the logic contract inside the entry contract and call a function in each storage contract to change the controller contract to the new logic contract. The old logic contract will be destructed. When upgrading a logic contract, new storage may be required. In this case a new storage contract will be created for the new logic contract. This storage contract will be given the address of the new logic contract as the controlling contract, and it shall be provided to the upgradeLogicContract function to be added to the array of storage contracts.

Prescription unique identifiers are used for generating the unique QR code discussed above. The QR code makes it possible for the pharmacist to both identify the prescription and spend the tokens associated with it. If a patient uses the same QR code at more than 1 pharmacy, the additional pharmacies won't be able to associate any new prescriptions on-chain, nor will they be able to spend any new tokens.

A biometric authentication device could be used to sign blockchain transactions. This would eliminate the need for additional digital signatures. However, if a device was lost or broken, a physician would lose access to their tokens tied to the blockchain address. This could be remedied by including an additional smart contract function that provides an additional signature from the biometric authentication device. A managed wallet service could be used by the patient instead of managing his/her own keys and data. Such a managed wallet service would be a trusted third party managing the blockchain compatible keys for the user.

Implementations eliminate the problem of asymmetric key management by removing the need for patients and physicians to necessarily generate and hold their own public-private keys. Instead, this management happens by the platform application similar to how hosted cryptocurrency wallets manage keys. However, users may want to use their own keys on their own devices, through secure chip components.

The tokens described above can be “fungible” tokens, i.e. tokens that can be replaced by another token. However, implementations can use “non-fungible tokens”, i.e. tokens that include unique information to render them distinguishable from other tokens. When using non-fungible tokens, the specific token(s), not merely a number or value of tokens, can be associated with a prescription to uniquely identify the prescription through the token itself. In such a case, the specific nonfungible toke(s) will be exchanged depending on the status of the prescription as disclosed above.

The token will best incentivize for pharmacies to use the platform, if the token has value. Value in this case means exchange rate to other cryptocurrencies or fiat currencies, such as US Dollars in this example. To create value, the platform can require that physicians and associated healthcare organizations have a certain amount of value in tokens in their accounts, as denominated in terms of USD, for example. This will create a base demand denominated in USD terms on the market. If the exchange rate of tokens falls relative to the USD, more tokens can be bought by each organization, creating natural demand pressure. If the token appreciates against the USD without a corresponding growth in organizational demand, it will stabilize back again. This way the exchange rate of tokens to USD should roughly correspond with the overall growth of the ecosystem.

As noted above, a pre-defined fraction of, or multiple of, tokens will be paired with each prescription being transmitted on the platform. The tokens will be utilized to facilitate prescription transmissions on the network, as well as being a native currency for payments inside the system. The token transaction fee utilized on the system can be adjusted up or down as determined by a governing body of the system. Imposing an economic cost on prescribing highly controlled drugs should encourage physicians to limit their prescribing of these drugs to only patients who absolutely need them. Organizations who employ physicians (or in the case of independent physicians, the physicians themselves) can be required to pay a small monthly subscription fee to use gain credentialed access to the system. If a healthcare organization fails to pay their monthly subscription fee, their access can be revoked. This ensures that all participants have a commitment to the system.

Pharmacies can also be required to pay a small subscription fee to gain credentialed access to the system. As noted above, for each prescription a pharmacy receives, the corresponding amount in tokens attached to that prescription will be deposited in the pharmacy's integrated wallet. However, pharmacies can also receive tokens from patients in the form of payments for goods and services received at the pharmacy. Instead of paying at the register for their prescriptions or other goods with cash or credit card, patients will use tokens that have been stored in their integrated cryptocurrency wallet. Pharmacies will be able to move tokens freely in and out of their wallets.

The system constitutes a completely new revenue stream for pharmacies, in that it can include direct payments from physicians to pharmacies. Conventionally, the only source of revenue for pharmacies was reimbursements from Pharmacy Benefit Management (PBM) companies, insurance companies, or co-payments from patients at the point of sale. Often, pharmacies must wait months for reimbursement from these entities, in addition to having extremely slim, or non-existent margins on their sale of drugs. Additionally, the platform provides a virtually fee-less payment channel that pharmacies may utilize to accept payments for goods and services from their patients. Controlled Drug prescriptions on the platform will also save pharmacists a great deal of time and energy that was previously spent on verifying the authenticity of paper prescriptions. The authenticity and source of prescriptions will no longer be in question.

Physicians, participating in the platform will re-gain complete control over their prescriptive authority of controlled drugs. This control can only be generated with the trustless platform and its non-invasive biometric identification methods. Additionally, physicians will have access to a complete, immutable record of prescriptions for their patients (even prescriptions written outside their healthcare system), allowing them to have complete knowledge of the patient's prescription history to better inform their prescribing practices. By preventing costly drug overdose ER visits and hospitalizations, encouraging safe and responsible opioid prescribing practices, various healthcare organizations will also see financial benefit.

The computing platforms may include one or more computer processors configured to execute computer program modules. The computer program modules may be configured to enable a user associated with the computing platform to interface with RX server 100 and/or external resources. By way of non-limiting example, the computing platforms may include one or more of a desktop computer, a laptop computer, a handheld computer, a tablet computing platform, a NetBook, a Smartphone, a gaming console, and/or other computing platforms. External resources may include sources of information outside of RX sever 100,

RX server 100 may include electronic storage, one or more processors and/or other components. RX server 100 may include communication lines, or ports to enable the exchange of information with a network and/or other computing platforms. RX server 100 may include a plurality of hardware, software, and/or firmware components operating together to provide the functionality attributed herein to RX server 100. For example, RX server 100 may be implemented by a cloud of computing platform operating together as RX server 100.

Electronic storage may include non-transitory storage media that electronically stores information, such as data and executable code. The electronic storage media may include one or both of system storage that is provided integrally (i.e., substantially non-removable) and/or removable storage Electronic storage may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage may include one or more virtual storage resources (e.g., cloud storage, a virtual private network, and/or other virtual storage resources). Electronic storage may store software algorithms, information and/or other information that enables RX server 100 and the computing platforms to function as described herein.

Processor(s) may be configured to provide information processing capabilities in RX server 100 and the computing platforms. As such, processor(s) may include one or more of a digital processor, an analog processor, a digital circuit designed to process information, an analog circuit designed to process information, a state machine, and/or other mechanisms for electronically processing information.

Processor(s) may be configured to execute modules by software; hardware; firmware; some combination of software, hardware, and/or firmware; and/or other mechanisms for configuring processing capabilities on processor(s). As used herein, the term “module” may refer to any component or set of components that perform the functionality attributed to the module. This may include one or more physical processors during execution of processor readable instructions, the processor readable instructions, circuitry, hardware, storage media, or any other components.

As noted above, any blockchain can be used in connection with the implementation. Further multiple blockchains could be used as long as some mechanism for reading information from each blockchain is provided. The various functions can be provided by smart contracts on the blockchain(s) or by conventional computing logic such as executable code or scripts.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the invention as defined by the appended claims. For example, it is to be understood that the present technology contemplates that, to the extent possible, one or more features of any implementation can be combined with one or more features of any other implementation.

What is claimed:

1. A computer implemented method for securely managing prescription records, the method comprising:
receiving, from a prescriber computing platform, a prescription request, wherein the prescription request includes prescription data describing a prescription:
approving the prescription request if the prescriber platform has been authenticated as corresponding to an authorized prescriber;
recording the prescription data in a database as a prescription record, wherein the prescription record includes a prescriber ID, a pharmacy ID, a patient ID, and a medication;
creating a cryptographic hash of at least some of the prescription data and auxiliary information to create a unique identifier for the prescription;
recording the hash as an issue transaction corresponding to the prescription;
receiving, from a pharmacy computing platform, an acceptance message indicating that the pharmacy platform is willing to fill the prescription;
verifying the accept message if the pharmacy platform corresponds to the pharmacy ID and the pharmacy is an authorized pharmacy; and
recording an accept transaction on a blockchain corresponding to the prescription, the accept transaction including the hash.

2. The method of claim 1, wherein the prescription request further includes a value in cryptographic tokens associated with the prescription data and further comprising transferring the value in cryptographic tokens from a cryptographic wallet of the prescriber to a cryptographic wallet of the pharmacy after said verifying the accept message.

3. The method of claim 1, further comprising dispensing the medication indicated in the prescription record by a pharmacy associated with the pharmacy ID to a patient associated with the patient ID.

4. The method of claim 2, wherein the cryptographic wallet of the prescriber can only transfer cryptographic tokens in association with a prescription.

5. The method of claim 1, wherein each prescription can exist only once on a blockchain.

6. The method of claim 1, wherein the prescriber ID, the pharmacy ID and the patient ID are recorded on a blockchain in association with the hash.

7. The method of claim 1, wherein, the prescriber ID, the pharmacy ID and the patient ID are associated with the hash in the database and wherein the database is not a blockchain.

8. The method of claim 1, wherein the patient ID is derived from a known unique string and a secret salt.

9. The method of claim 8, wherein the known unique string is a social security number of the patient.

10. The method of claim 1, further comprising printing a visible code that indicates the unique identifier and transmitting data indicating the visible code to a patient computing platform.

11. A computer system securely managing prescription records, the method comprising:
at least one computer hardware processor; and
at least one computer memory storing instructions thereon which, when executed by the at least one computer hardware processor, cause the at least one computer hardware processor to:
receive, from a prescriber computing platform, a prescription request, wherein the prescription request includes prescription data describing a prescription;
approve the prescription request if the prescriber platform has been authenticated as corresponding to an authorized prescriber;
record the prescription data in a database as a prescription record, wherein the prescription record includes a prescriber ID, a pharmacy ID, a patient ID, and a medication;
create a cryptographic hash of at least some of the prescription data and auxiliary information to create a unique identifier for the prescription;
record the hash as an issue transaction corresponding to the prescription;
receive, from a pharmacy computing platform, an acceptance message indicating that the pharmacy platform is willing to fill the prescription;
verify the accept message if the pharmacy platform corresponds to the pharmacy ID and the pharmacy is an authorized pharmacy; and
record an accept transaction on a blockchain corresponding to the prescription, the accept transaction including the hash.

12. The system of claim 11, wherein the prescription request further includes a value in cryptographic tokens associated with the prescription data and further comprising transferring the value in cryptographic tokens from a cryptographic wallet of the prescriber to a cryptographic wallet of the pharmacy after said verifying the accept message.

13. The system of claim 11, wherein the medication indicated in the prescription record is dispensed by a pharmacy associated with the pharmacy ID to a patient associated with the patient ID.

14. The system of claim 12, wherein the cryptographic wallet of the prescriber can only transfer cryptographic tokens in association with a prescription.

15. The system of claim 11, wherein each prescription can exist only once on a blockchain.

16. The system of claim 1, wherein the prescriber ID, the pharmacy ID and the patient ID are recorded on a blockchain in association with the hash.

17. The system of claim 11, wherein, the prescriber ID, the pharmacy ID and the patient ID are associated with the hash in the database and wherein the database is not a blockchain.

18. The system of claim 11, wherein the patient ID is derived from a known unique string and a secret salt.

19. The system of claim 18, wherein the known unique string is a social security number of the patient.

20. The system of claim 1, wherein the instructions further cause the ate least one computer hardware processor to print a visible code that indicates the unique identifier and transmitting data indicating the visible code to a patient computing platform.
